(19) United States
(12) Patent Application Publication
(10) Pub. No.: US 2011/0124903 A1
(43) Pub. Date: May 26, 2011

(54) SOLID STATE FORMS OF FESOTERODINE INTERMEDIATES

(75) Inventors: Kishore CHARUGUNDLA, Andhra Pradesh (IN); Udhaya KUMAR, Pondicherry (IN); Neela Praveen KUMAR, Andhra Pradesh (IN); Nitin Sharadchandra PRADHAN, Maharashtra (IN)

(73) Assignee: ACTAVIS GROUP PTC EHF, Hafnarfjordur (IS)

(21) Appl. No.: 12/949,102

(22) Filed: Nov. 18, 2010

(30) Foreign Application Priority Data
Nov. 20, 2009 (IN) 2856/CHE/2009

(51) Int. Cl. C07C 69/017; C07C 211/27

(52) U.S. Cl. 560/140; 564/316

(57) ABSTRACT

Provided herein are novel solid state forms of fesoterodine intermediates, (R)-4-benzyloxy-3-(3-diisopropylamino-1-phenylpropyl)-benzoic acid and (R)-[4-benzyloxy-3-(3-diisopropylamino-1-phenylpropyl)-phenyl]-methanol, and processes for their preparation thereof. The solid state intermediates are useful for preparing fesoterodine or a pharmaceutically acceptable salt thereof in high purity.

### CROSS REFERENCE TO RELATED APPLICATION

This application claims the benefit of priority to Indian provisional application No. 2856/CHE/2009, filed on Nov. 20, 2009, which is incorporated herein by reference in its entirety.

### FIELD OF THE DISCLOSURE

Provided herein are novel solid state forms of fesoterodine intermediates and processes for their preparation thereof. The solid state intermediates are useful for preparing fesoterodine or a pharmaceutically acceptable salts thereof in high yield and purity.

### BACKGROUND

U.S. Pat. No. 6,713,464 B1 discloses a variety of 3,3-diphenylpropylamine derivatives, processes for their preparation, pharmaceutical compositions comprising the derivatives, and methods of use thereof. These compounds are anti-muscarinic agents with superior pharmacokinetic properties compared to existing drugs such as oxybutynin and tolterodine, which are useful in the treatment of urinary incontinence, gastrointestinal hyperactivity (irritable bowel syndrome) and other smooth muscle contractile conditions. Among them, fesoterodine, chemically 2-[(1R)-3-[bis(1-methylethyl)amino]-1-phenylpropyl]-4-hydroxymethylphenyl isobutyrate is a new, potent and competitive muscarinic antagonist and useful in the potential treatment of urinary incontinence. Fesoterodine is represented by the following structural formula I:

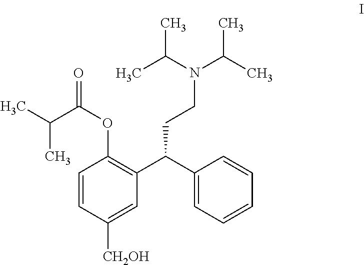

Processes for the preparation of fesoterodine and related compounds, and their pharmaceutically acceptable salts, are disclosed in the U.S. Pat. Nos. 6,713,464 B1 and 6,858,650 B1; U.S. Patent Application No. 2006/0270738 and PCT Publication Nos. WO 2007/138440 A1 and WO 2009/037569 A2.

According to U.S. Pat. No. 6,713,464 B1 (hereinafter referred to as the '464 patent), fesoterodine is prepared by the reaction of (±)-6-bromo-4-phenylchroman-2-one with benzyl chloride in the presence of sodium iodide and anhydrous potassium carbonate in methanol and acetone, to give (±)-3-(2-benzyloxy-5-bromophenyl)-3-phenylpropionic acid methyl ester as a light yellow oil, which by reduction with lithium aluminium hydride in tetrahydrofuran at room temperature (reaction time: 18 hours) produces (±)-3-(2-benzyloxy-5-bromophenyl)-3-phenylpropan-1-ol. (±)-3-(2-Benzyloxy-5-bromophenyl)-3-phenylpropan-1-ol is then treated with p-toluenesulphonyl chloride in the presence of pyridine in dichloromethane to afford (±)-toluene-4-sulphonic acid 3-(2-benzyloxy-5-bromophenyl)-3-phenylpropyl ester, followed by reaction with N,N-diisopropylamine in acetonitrile at reflux temperature (i.e., 75-80° C.) for 97 hours to produce (±)-[3-(2-benzyloxy-5-bromophenyl)-3-phenylpropyl]-diisopropylamine as a brown and viscous syrup, which is resolved to produce (R)-[3-(2-benzyloxy-5-bromophenyl)-3-phenylpropyl]-diisopropylamine, which is then subjected to a Grignard reaction with ethyl bromide and magnesium in the presence of solid carbon dioxide in tetrahydrofuran to produce (R)-4-benzyloxy-3-(3-diisopropylamino-1-phenylpropyl)-benzoic acid hydrochloride. Esterification of (R)-4-benzyloxy-3-(3-diisopropylamino-1-phenylpropyl)-benzoic acid hydrochloride with methanol in the presence of sulphuric acid produces (R)-4-benzyloxy-3-(3-diisopropylamino-1-phenylpropyl)-benzoic acid methyl ester, which is then reduced with lithium aluminium hydride (reaction time: 18 hours) to produce (R)-[4-benzyloxy-3-(3-diisopropylamino-1-phenylpropyl)-phenyl]-methanol, which is then subjected to deprotection with Raney-Nickel to produce (R)-2-(3-diisopropylamino-1-phenylpropyl)-4-hydroxymethylphenol, followed by condensation with isobutyryl chloride in an inert solvent in the presence of a base to give fesoterodine.

Fesoterodine obtained by the processes described in the above prior art does not have satisfactory purity for pharmaceutical use. Unacceptable amounts of impurities are generally formed along with fesoterodine. In addition, the processes involve the additional step of column chromatographic purifications or multiple crystallizations. Methods involving column chromatographic purifications are generally undesirable for large-scale operations as they require additional expensive setup, adding to the cost of production, thereby making the processes commercially unfeasible.

In the preparation of fesoterodine, (R)-4-benzyloxy-3-(3-diisopropylamino-1-phenylpropyl)-benzoic acid of formula A, and (R)-[4-benzyloxy-3-(3-diisopropylamino-1-phenylpropyl)-phenyl]-methanol of formula B:

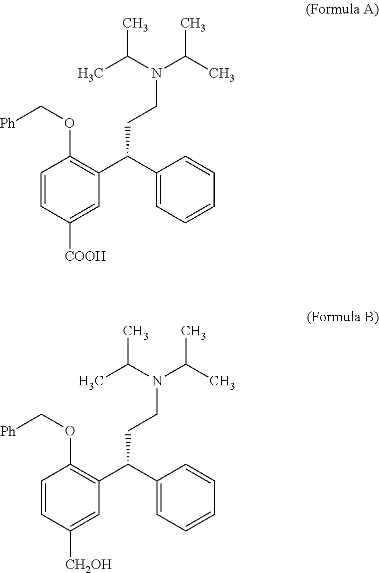

are key intermediates. Solid state forms of the intermediate compounds formulae A & B have not been reported in the literature. The main draw back of the processes described prior art is that the alkylating agents like benzyl chloride and ethyl bromide, which are classified as genotoxic compounds, are formed as impurities in the respective intermediates. These impurities carry forward during the synthesis of fesoterodine and they remain in the fesoterodine as persistent impurities. It is difficult to remove these persistent and genotoxic impurities in the fesoterodine or a pharmaceutically acceptable salt thereof at final stages, even after carrying out extensive and lengthy purification processes.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in fesoterodine or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards have been set with the intention of ensuring that an API is as free of impurities as possible, and, thus, are as safe as possible for clinical use. The United States Food and Drug Administration guidelines recommend that the amounts of some impurities limited to less than 0.1 percent.

Hence, a need still remains for improved and commercially viable processes of isolating fesoterodine intermediates as solid state forms that will solve the aforesaid problems associated with the process described in the prior art and will be suitable for large-scale preparation, in terms of simplicity, purity and yield of the product.

### SUMMARY

Extensive research and experimentation was carried out by the present inventors to eliminate or completely remove the traces of genotoxic impurities such as benzyl chloride and ethyl bromide, formed during the synthesis, in the fesoterodine intermediates. As a result, it has been found that the content of these genotoxic impurities and other byproducts such as isopropyl tosylate and methyl tosylate, formed in the synthesis of the fesoterodine intermediates, can be substantially removed by the isolation processes, to produce solid states forms, disclosed herein.

Solid state forms of fesoterodine intermediates, specifically (R)-4-benzyloxy-3-(3-diisopropylamino-1-phenylpropyl)-benzoic acid of formula A and (R)-[4-benzyloxy-3-(3-diisopropylamino-1-phenylpropyl)-phenyl]-methanol of formula B, have not been reported, isolated, or characterized in the literature. The present inventors have surprisingly and unexpectedly found that the intermediate compounds (R)-4-benzyloxy-3-(3-diisopropylamino-1-phenylpropyl)-benzoic acid and (R)-[4-benzyloxy-3-(3-diisopropylamino-1-phenylpropyl)-phenyl]-methanol, can be isolated in pure solid state forms.

In one aspect, the fesoterodine intermediates in a crystalline form are provided. In yet another aspect, the fesoterodine intermediates in an amorphous form are provided.

It has also been found that the solid state forms of fesoterodine intermediates are useful intermediates in the preparation of fesoterodine or a pharmaceutically acceptable salt thereof, preferably fesoterodine fumarate, in high purity.

### BRIEF DESCRIPTION OF THE DRAWINGS

FIG. 1 is a characteristic powder X-ray diffraction (XRD) pattern of an amorphous form of (R)-4-benzyloxy-3-(3-diisopropylamino-1-phenylpropyl)-benzoic acid.

FIG. 2 is a characteristic infra red (IR) spectrum of an amorphous form of (R)-4-benzyloxy-3-(3-diisopropylamino-1-phenylpropyl)-benzoic acid.

FIG. 3 is a characteristic differential scanning calorimetric (DSC) thermogram of an amorphous form of (R)-4-benzyloxy-3-(3-diisopropylamino-1-phenylpropyl)-benzoic acid.

FIG. 4 is a characteristic powder X-ray diffraction (XRD) pattern of a crystalline form of (R)-4-benzyloxy-3-(3-diisopropylamino-1-phenylpropyl)-benzoic acid.

FIG. 5 is a characteristic infra red (IR) spectrum of a crystalline form of (R)-4-benzyloxy-3-(3-diisopropylamino-1-phenylpropyl)-benzoic acid.

FIG. 6 is a characteristic differential scanning calorimetric (DSC) thermogram of a crystalline form of (R)-4-benzyloxy-3-(3-diisopropylamino-1-phenylpropyl)-benzoic acid.

FIG. 7 is a characteristic powder X-ray diffraction (XRD) pattern of a crystalline form of (R)-[4-benzyloxy-3-(3-diisopropylamino-1-phenylpropyl)-phenyl]-methanol.

FIG. 8 is a characteristic infra red (IR) spectrum of a crystalline form of (R)-[4-benzyloxy-3-(3-diisopropylamino-1-phenylpropyl)-phenyl]-methanol.

FIG. 9 is a characteristic differential scanning calorimetric (DSC) thermogram of a crystalline form of (R)-[4-benzyloxy-3-(3-diisopropylamino-1-phenylpropyl)-phenyl]-methanol.

### DETAILED DESCRIPTION

According to one aspect, provided herein are solid state forms of fesoterodine intermediates, wherein the fesoterodine intermediate is selected from the group consisting of (R)-4-benzyloxy-3-(3-diisopropylamino-1-phenylpropyl)-benzoic acid of formula A and (R)-[4-benzyloxy-3-(3-diisopropylamino-1-phenylpropyl)-phenyl]-methanol of formula B:

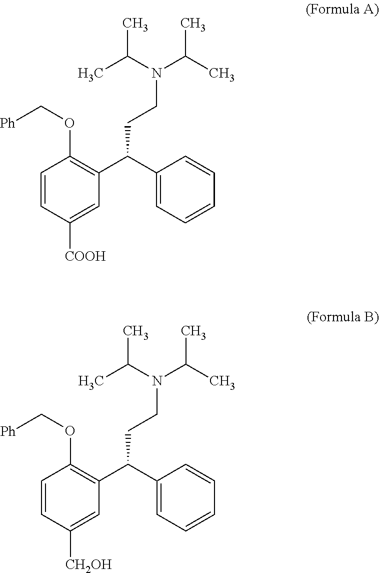

In one embodiment, the solid state forms of fesoterodine intermediates exist in a crystalline form. In another embodiment, the solid state forms of fesoterodine intermediates exist in an amorphous form.

In one embodiment, the solid state forms of fesoterodine intermediates have the following characteristics, wherein:

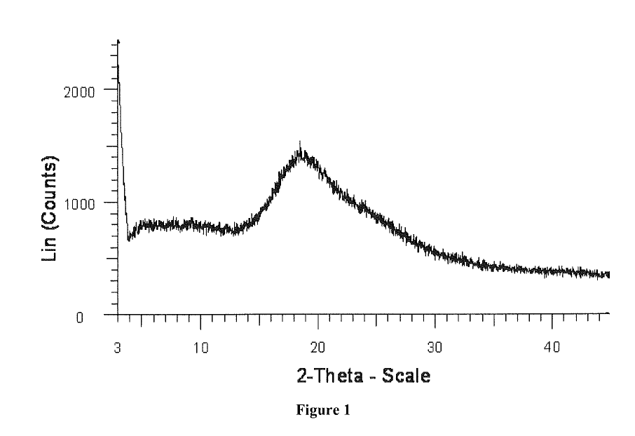

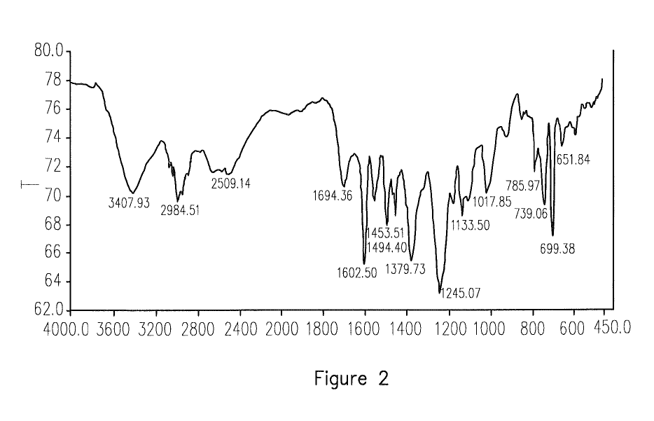

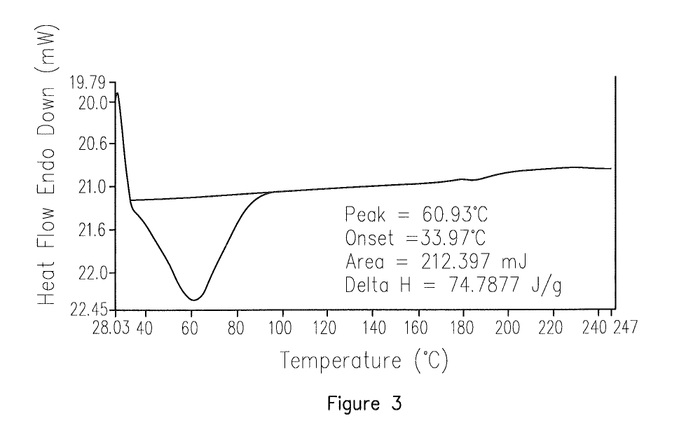

a) the solid state form of (R)-4-benzyloxy-3-(3-diisopropylamino-1-phenylpropyl)-benzoic acid is an amorphous form characterized by one or more of the following properties:
i) a powder X-ray diffraction pattern showing no peaks substantially in accordance with FIG. 1;
ii) an IR spectrum substantially in accordance with FIG. 2;
iii) an IR spectrum having absorption bands at 3407, 2984, 2509, 1694, 1602, 1550, 1494, 1453, 1379, 1245, 1133, 1017, 785, 739, 699 and 651±1 cm⁻¹; and
iv) a differential scanning calorimetric (DSC) thermogram substantially in accordance with FIG. 3;

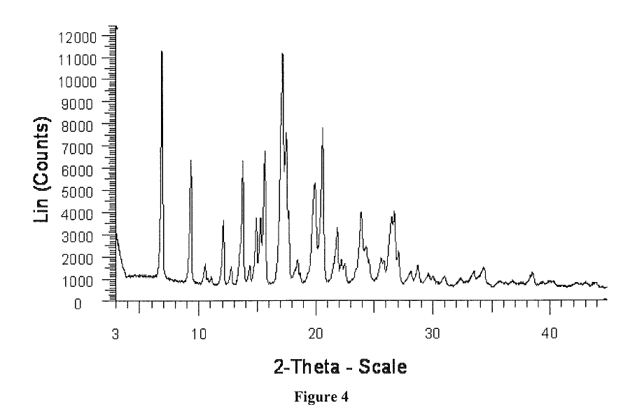

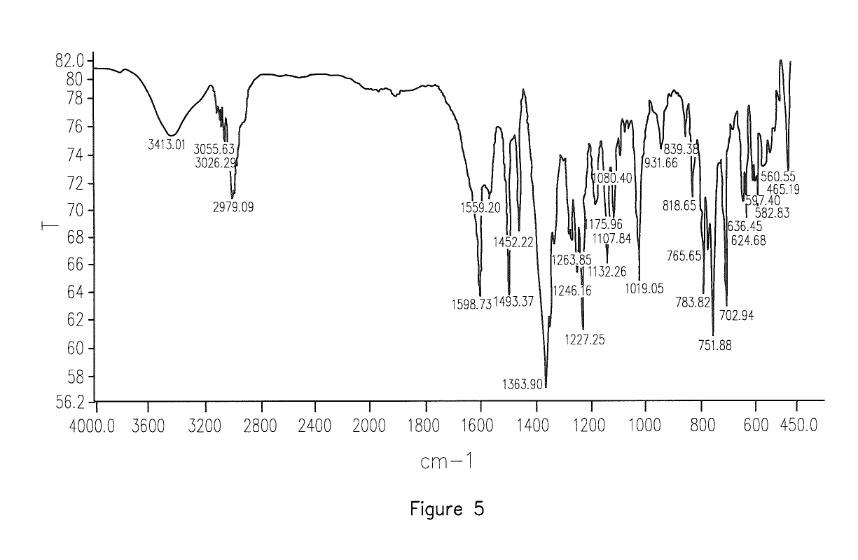

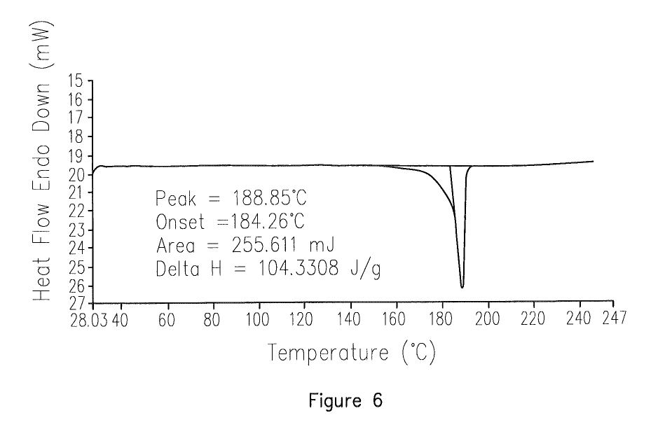

b) the solid state form of (R)-4-benzyloxy-3-(3-diisopropylamino-1-phenylpropyl)-benzoic acid is a crystalline form characterized by one or more of the following properties:
i) a powder X-ray diffraction pattern substantially in accordance with FIG. 4;
ii) a powder X-ray diffraction pattern having peaks at about 6.82, 9.27, 12.06, 13.44, 13.73, 14.88, 15.25, 15.62, 17.14, 17.45, 17.68, 19.92, 20.59, 21.83, 23.89, 24.30, 26.49, 26.72 and 27.06±0.2 degrees 2-theta;
iii) an IR spectrum substantially in accordance with FIG. 5; and
iv) an IR spectrum having absorption bands at 3413, 3055, 3026, 2979, 1598, 1559, 1493, 1452, 1363, 1263, 1246, 1227, 1132, 1019, 931, 783, 751, 702 and 624±1 cm⁻¹; and
v) a differential scanning calorimetric (DSC) thermogram substantially in accordance with FIG. 6; and

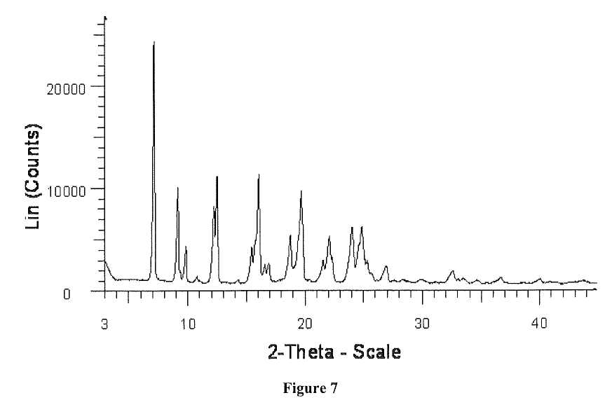

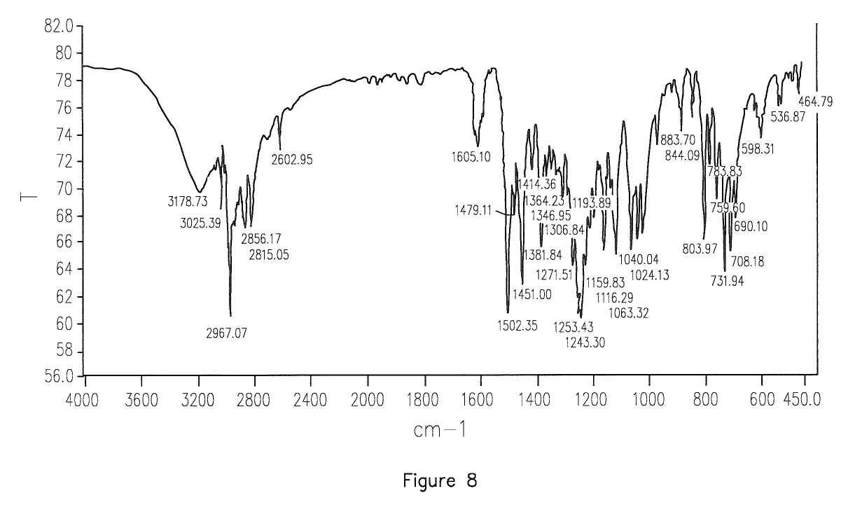

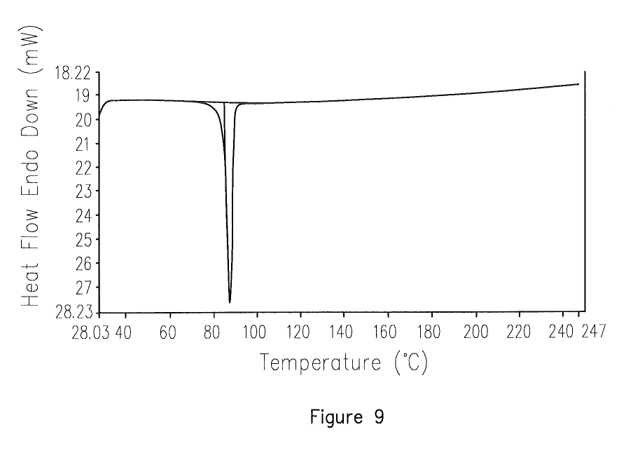

c) the solid state form of (R)-[4-benzyloxy-3-(3-diisopropylamino-1-phenylpropyl)-phenyl]-methanol is a crystalline form characterized by one or more of the following properties:
i) a powder X-ray diffraction pattern substantially in accordance with FIG. 7;
ii) a powder X-ray diffraction pattern having peaks at about 7.07, 9.09, 9.76, 12.17, 12.43, 15.41, 15.71, 18.73, 19.66, 22.05, 22.32, 24.0, 24.57 and 24.84±0.2 degrees 2-theta;
iii) an IR spectrum substantially in accordance with FIG. 8;
iv) an IR spectrum having absorption bands at 3178, 3025, 2967, 2856, 2815, 1605, 1502, 1479, 1451, 1384, 1271, 1253, 1243, 1159, 1116, 1063, 1040, 1024, 803, 731, 708 and 691±1 cm⁻¹; and
v) a differential scanning calorimetric (DSC) thermogram substantially in accordance with FIG. 9.

The solid state forms of fesoterodine intermediates are stable, consistently reproducible, and are particularly suitable for bulk preparation and handling. Moreover, the solid state forms of (R)-4-benzyloxy-3-(3-diisopropylamino-1-phenylpropyl)-benzoic acid and (R)-[4-benzyloxy-3-(3-diisopropylamino-1-phenylpropyl)-phenyl]-methanol are useful intermediates in the preparation of fesoterodine or a pharmaceutically acceptable salt thereof, specifically fesoterodine fumarate, in high purity.

According to another aspect, there is provided a process for the preparation of an amorphous form of (R)-4-benzyloxy-3-(3-diisopropylamino-1-phenylpropyl)-benzoic acid, comprising:

a) providing a solution of (R)-4-benzyloxy-3-(3-diisopropylamino-1-phenylpropyl)-benzoic acid in a chlorinated hydrocarbon solvent;

b) optionally, filtering the solution to remove extraneous matter; and

c) substantially removing the chlorinated hydrocarbon solvent from the solution to provide the amorphous form of (R)-4-benzyloxy-3-(3-diisopropylamino-1-phenylpropyl)-benzoic acid.

The term “substantially removing” the solvent refers to at least 80%, specifically greater than about 85%, more specifically greater than about 90%, still more specifically greater than about 99%, and most specifically essentially complete (100%), removal of the solvent from the solvent solution.

The process can produce an amorphous form of (R)-4-benzyloxy-3-(3-diisopropylamino-1-phenylpropyl)-benzoic acid in substantially pure form.

The term “substantially pure amorphous (R)-4-benzyloxy-3-(3-diisopropylamino-1-phenylpropyl)-benzoic acid” refers to the amorphous (R)-4-benzyloxy-3-(3-diisopropylamino-1-phenylpropyl)-benzoic acid having a purity of greater than about 95%, specifically greater than about 96%, more specifically greater than about 97%, and still more specifically greater than about 99% (measured by HPLC).

The amorphous (R)-4-benzyloxy-3-(3-diisopropylamino-1-phenylpropyl)-benzoic acid obtained by the process disclosed herein can be converted into a crystalline form of (R)-4-benzyloxy-3-(3-diisopropylamino-1-phenylpropyl)-benzoic acid as per the process disclosed hereinafter.

Exemplary chlorinated hydrocarbon solvents used in step-(a) include, but are not limited to, methylene chloride, ethylene dichloride, chloroform, carbon tetrachloride, and mixtures thereof. A specific chlorinated hydrocarbon solvent is methylene chloride.

Step-(a) of providing a solution of (R)-4-benzyloxy-3-(3-diisopropylamino-1-phenylpropyl)-benzoic acid includes dissolving or extracting the (R)-4-benzyloxy-3-(3-diisopropylamino-1-phenylpropyl)-benzoic acid in the chlorinated hydrocarbon solvent, or obtaining an existing solution from a previous processing step.

In one embodiment, the (R)-4-benzyloxy-3-(3-diisopropylamino-1-phenylpropyl)-benzoic acid is dissolved or extracted in the chlorinated hydrocarbon solvent at a temperature of 0° C. to the boiling temperature of the solvent used, specifically at about 20° C. to about 100° C., and more specifically at about 25° C. to about 80° C.

In another embodiment, the solution in step-(a) is prepared by reacting (R)—N,N-diisopropyl-3-(2-benzyloxy-5-bromophenyl)-3-phenylpropylamine with a mixture of ethyl bromide, iodine and magnesium in a solvent to produce a first reaction mass; treating the first reaction mass with solid carbon dioxide, optionally in the presence of a Lewis acid, to produce a second reaction mass containing (R)-4-benzyloxy-3-(3-diisopropylamino-1-phenylpropyl)-benzoic acid; followed by usual work up such as a filtration, a washing, an extraction, an acid/base treatment, an evaporation, or a combination thereof. In one embodiment, the work-up includes dissolving or extracting the resulting (R)-4-benzyloxy-3-(3-diisopropylamino-1-phenylpropyl)-benzoic acid in the chlorinated hydrocarbon solvent at a temperature of 0° C. to the boiling temperature of the solvent used, specifically at about 20° C. to about 100° C., and more specifically at about 25° C. to about 80° C.

Exemplary solvents suitable for facilitating the reaction between (R)—N,N-diisopropyl-3-(2-benzyloxy-5-bromophenyl)-3-phenylpropylamine with the mixture of ethyl bromide, iodine and magnesium include, but are not limited to, acetonitrile, tetrahydrofuran, dimethylformamide, dimethylsulfoxide, dioxane, and mixtures thereof. A specific solvent is tetrahydrofuran. In one embodiment, the reaction is carried out at a temperature of about −65° C. to the reflux temperature of the solvent used, specifically at a temperature of about −65° C. to about 100° C. for at least 30 minutes, and most specifically at a temperature of about −60° C. to about 60° C. for about 1 hour to about 6 hours.

Alternatively, the solution in step-(a) is prepared by treating an acid addition salt of (R)-4-benzyloxy-3-(3-diisopropylamino-1-phenylpropyl)-benzoic acid with a base to liberate (R)-4-benzyloxy-3-(3-diisopropylamino-1-phenylpropyl)-benzoic acid free base and dissolving or extracting the (R)-4-benzyloxy-3-(3-diisopropylamino-1-phenylpropyl)-benzoic acid in the chlorinated hydrocarbon solvent.

The acid addition salts are derived from a therapeutically acceptable acid such as hydrochloric acid, hydrobromic acid, sulfuric acid, nitric acid, oxalic acid, acetic acid, propionic acid, phosphoric acid, succinic acid, maleic acid, fumaric acid, citric acid, tartaric acid, di-p-toluoyl-L-(+)-tartaric acid, malic acid, and ascorbic acid. A specific acid addition salt of (R)-4-benzyloxy-3-(3-diisopropylamino-1-phenylpropyl)-benzoic acid is hydrochloride salt.

The treatment of an acid addition salt with base is carried out in a solvent selected from the group consisting of water, an ester, an alcohol, a ketone, a chlorinated hydrocarbon, a hydrocarbon, an ether, and mixtures thereof.

The base can be an inorganic or an organic base. Specific organic bases are triethyl amine, tributylamine, diisopropylethylamine, diethylamine, tert-butyl amine, N-methylmorpholine, pyridine, 4-(N,N-dimethylamino)pyridine. Exemplary inorganic bases include, but are not limited to, hydroxides, carbonates and bicarbonates of alkali or alkaline earth metals. Specific inorganic bases are sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, and more specifically sodium bicarbonate, sodium hydroxide, potassium hydroxide, sodium carbonate and potassium carbonate.

The solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing (R)-4-benzyloxy-3-(3-diisopropylamino-1-phenylpropyl)-benzoic acid by removing charcoal or silica gel. Preferably, finely powdered carbon is an active carbon. A specific mesh size of silica gel is 40-500 mesh, and more specifically 60-120 mesh.

Removal of solvent in step-(c) is accomplished, for example, by substantially complete evaporation of the solvent, concentrating the solution or distillation of solvent, under inert atmosphere to obtain amorphous (R)-4-benzyloxy-3-(3-diisopropylamino-1-phenylpropyl)-benzoic acid.

In one embodiment, the solvent is removed by evaporation. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

In one embodiment, the amorphous (R)-4-benzyloxy-3-(3-diisopropylamino-1-phenylpropyl)-benzoic acid obtained in step-(c) is recovered by methods such as filtration, filtration under vacuum, decantation, centrifugation, or a combination thereof. In one embodiment, the amorphous (R)-4-benzyloxy-3-(3-diisopropylamino-1-phenylpropyl)-benzoic acid is recovered by filtration employing a filtration media of, for example, a silica gel or celite.

According to another aspect, there is provided a process for the preparation of a crystalline form of (R)-4-benzyloxy-3-(3-diisopropylamino-1-phenylpropyl)-benzoic acid, comprising:

a) providing a solution of (R)-4-benzyloxy-3-(3-diisopropylamino-1-phenylpropyl)-benzoic acid in an alcohol solvent;

b) optionally, filtering the solution to remove extraneous matter; and

c) isolating and/or recovering crystalline form of (R)-4-benzyloxy-3-(3-diisopropylamino-1-phenylpropyl)-benzoic acid from the solution.

The process can produce a crystalline form of (R)-4-benzyloxy-3-(3-diisopropylamino-1-phenylpropyl)-benzoic acid in substantially pure form.

The term “substantially pure crystalline form of (R)-4-benzyloxy-3-(3-diisopropylamino-1-phenylpropyl)-benzoic acid” refers to the crystalline form of (R)-4-benzyloxy-3-(3-diisopropylamino-1-phenylpropyl)-benzoic acid having a purity of greater than about 98%, specifically greater than about 99%, more specifically greater than about 99.5%, and still more specifically greater than about 99.9%. The purity is preferably measured by High Performance Liquid Chromatography (HPLC). For example, the purity of the crystalline form of (R)-4-benzyloxy-3-(3-diisopropylamino-1-phenylpropyl)-benzoic acid obtained by the process disclosed herein can be about 98% to about 99%, or about 99.5% to about 99.9%, as measured by HPLC.

In one embodiment, the crystalline form of (R)-4-benzyloxy-3-(3-diisopropylamino-1-phenylpropyl)-benzoic acid obtained by the process disclosed herein is stable and consistently reproducible.

Exemplary alcohol solvents used in step-(a) include, but are not limited to, methanol, ethanol, n-propanol, isopropyl alcohol, isobutanol, n-butanol, tert-butanol, amyl alcohol, isoamyl alcohol, and mixtures thereof. The term solvent also includes mixtures of solvents. Specific alcohol solvents are methanol, ethanol, isopropyl alcohol, and mixtures thereof; and more specifically isopropyl alcohol.

Step-(a) of providing a solution of (R)-4-benzyloxy-3-(3-diisopropylamino-1-phenylpropyl)-benzoic acid includes dissolving or extracting the (R)-4-benzyloxy-3-(3-diisopropylamino-1-phenylpropyl)-benzoic acid in the alcohol solvent, or obtaining an existing solution from a previous processing step.

In one embodiment, the (R)-4-benzyloxy-3-(3-diisopropylamino-1-phenylpropyl)-benzoic acid is dissolved or extracted in the alcohol solvent at a temperature of 0° C. to the boiling temperature of the solvent used, specifically at about 40° C. to about 100° C., and more specifically at about 50° C. to about 80° C. In another embodiment, the amorphous form of (R)-4-benzyloxy-3-(3-diisopropylamino-1-phenylpropyl)-benzoic acid is also used as starting material to prepare the solution in step-(a).

In another embodiment, the solution in step-(a) can also be prepared by the processes as described above.

The solution obtained in step-(a) is optionally subjected to carbon treatment or silica gel treatment by the methods as described hereinabove.

The isolation of pure crystalline form of (R)-4-benzyloxy-3-(3-diisopropylamino-1-phenylpropyl)-benzoic acid in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method known in the art such as cooling, seeding, partial removal of the solvent from the solution, by adding an anti-solvent to the solution, or a combination thereof.

In one embodiment, the crystallization is carried out by cooling the solution while stirring at a temperature of below 35° C. for at least 15 minutes, specifically at about 0° C. to about 30° C. for about 30 minutes to about 20 hours.

The recovering in step-(c) is carried out by the methods as described hereinabove.

Fesoterodine or a pharmaceutically acceptable salt thereof, preferably fesoterodine fumarate, can be prepared in high purity by using the crystalline form of (R)-4-benzyloxy-3-(3-diisopropylamino-1-phenylpropyl)-benzoic acid obtained by the process disclosed herein, by the methods disclosed herein.

According to another aspect, there is provided a process for the preparation of a crystalline form of (R)-[4-benzyloxy-3-(3-diisopropylamino-1-phenylpropyl)-phenyl]-methanol, comprising:

a) reacting methyl (R)-4-benzyloxy-3-(3-diisopropylamino-1-phenylpropyl)-benzoate with a reducing agent, optionally in the presence of a Lewis acid, in a first solvent to produce a first reaction mass containing (R)-[4-benzyloxy-3-(3-diisopropylamino-1-phenylpropyl)-phenyl]-methanol;

b) optionally, subjecting the first reaction mass to work up methods selected from the group consisting of a filtration, a washing, an extraction, an acid/base treatment, an evaporation, or a combination thereof;

c) substantially removing the first solvent from the first reaction mass to provide an oily mass containing (R)-[4-benzyloxy-3-(3-diisopropylamino-1-phenylpropyl)-phenyl]-methanol;

d) combining the oily mass with a second solvent to produce a second reaction mass; and

e) recovering the crystalline form of (R)-[4-benzyloxy-3-(3-diisopropylamino-1-phenylpropyl)-phenyl]-methanol from the second reaction mass.

The process can produce a crystalline form of (R)-[4-benzyloxy-3-(3-diisopropylamino-1-phenylpropyl)-phenyl]-methanol in substantially pure form.

The term “substantially pure crystalline form of (R)-[4-benzyloxy-3-(3-diisopropylamino-1-phenylpropyl)-phenyl]-methanol” refers to the crystalline form of (R)-[4-benzyloxy-3-(3-diisopropylamino-1-phenylpropyl)-phenyl]-methanol having a purity of greater than about 98%, specifically greater than about 99%, more specifically greater than about 99.5%, and still more specifically greater than about 99.9% as measured by HPLC.

In one embodiment, the reducing agents used in step-(a) include, but are not limited to, lithium aluminium hydride, sodium borohydride, sodium cyanoborohydride and sodium triacetoxyborohydride.

Exemplary Lewis acids used in step-(a) include, but are not limited, aluminium chloride, calcium chloride, boron triflouride and zinc chloride. A specific Lewis acid is aluminium chloride.

Exemplary first solvents used in step-(a) include, but are not limited to, monoglyme, diglyme, aprotic solvents like tetrahydrofuran, ethers, and mixtures thereof. A specific first solvent is tetrahydrofuran.

The reaction in step-(a) is carried out at a temperature of about −20° C. to about 50° C., specifically at about 0° C. to about 40° C., and more specifically at about 20° C. to about 35° C.

Removal of solvent in step-(c) is accomplished by methods as described hereinabove, for example, by substantially complete evaporation of the solvent, concentrating the solution or distillation of solvent, under inert atmosphere.

In one embodiment, the second solvent used in step-(d) includes, but is not limited to, n-pentane, n-hexane, n-heptane, n-octane, petroleum ether, cyclohexane, and mixtures thereof. Specifically, the second solvent is selected from the group consisting of n-hexane, petroleum ether, cyclohexane, and mixtures thereof, and more specifically petroleum ether.

Combining the oily mass with the second solvent in step-(d) is done in a suitable order, for example, the oily mass is added to the second solvent, or alternatively, the second solvent is added to the oily mass. The addition is, for example, carried out drop wise or in one portion or in more than one portion. The addition is specifically carried out at a temperature of about 0° C. to about 90° C., and more specifically at about 20° C. to about 80° C. under stirring. After completion of the addition process, the resulting mass is stirred at a temperature of about 0° C. to about 50° C. for at least 30 minutes, specifically at about 10° C. to about 45° C. for about 1 hour to about 25 hours, and more specifically at a temperature of about 20° C. to about 40° C. for about 10 hours to about 20 hours to produce the second reaction mass.

The recovering in step-(e) is carried out by the methods as described hereinabove.

Fesoterodine or a pharmaceutically acceptable salt thereof, preferably fesoterodine fumarate, can be prepared in high purity by using the crystalline form of (R)-[4-benzyloxy-3-(3-diisopropylamino-1-phenylpropyl)-phenyl]-methanol obtained by the process disclosed herein, by the methods disclosed herein.

### Instrumental Details:

### X-Ray Powder Diffraction (P-XRD):

The X-Ray powder diffraction was measured by an X-ray powder diffractometer equipped with a Cu-anode (λ=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2-theta; step width=0.01579°; and measuring time per step=0.11 second.

### Infra Red (FT-IR) Spectroscopy:

FT-IR spectroscopy was performed with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

### Differential Scanning Calorimetry (DSC):

DSC (Differential Scanning calorimetry) measurements were performed with a Differential Scanning calorimeter (Diamond DSC, Perkin-Elmer) at a scan rate of 5° C. per minute. The nitrogen gas purge was at 40 ml/min. The instrument was calibrated for temperature and heat flow using indium as standards. The samples were encapsulated in to closed aluminium pans without hole subsequently crimped to ensure a tight seal. Data acquisition and analysis were performed using pyris software.

Aptly the processes described herein are adapted to the preparation of fesoterodine or a pharmaceutically acceptable salt thereof in high enantiomeric and chemical purity.

The following examples are given for the purpose of illustrating the present disclosure and should not be considered as limitation on the scope or spirit of the disclosure.

### Example

### Preparation of Fesoterodine Fumarate

### Step-1: Preparation of 6-Bromo-4-phenylchroman-2-one

Cinnamic acid (100 g, 676 mmol), 4-bromophenol (123 g, 730 mmol) and sulfuric acid (13 ml) were taken into a 1 L 4-neck round bottom flask. The contents were slowly heated to 120-125° C., and stirred for 3 to 4 hours at 120-125° C. The reaction mixture was cooled to 80° C., followed by the addition of toluene (300 ml) and water (200 ml), and then the mixture was stirred for 15 minutes. The toluene layer was separated and washed with water (2×100 ml). The resulting toluene layer was distilled completely under vacuum. Potassium carbonate solution (47% w/v-100 ml) was added to the residue at 25-30° C., the contents were stirred for 15 minutes and filtered the solid and washed with water (2×100 ml). The wet material was leached with 100 ml of isopropyl alcohol and then filtered. The resulting solid was washed with 50 ml of isopropyl alcohol and then the material was dried at 70-75° C. to give 72 g of 6-bromo-4-phenylchroman-2-one (Melting poing: 117° C.; HPLC Purity: 98.5%).

### Step-2: Preparation of Methyl (±)-3-(2-benzyloxy-5-bromophenyl)-3-phenyl Propionate

6-Bromo-4-phenylchroman-2-one (100 g, 330 mmole), potassium carbonate (55.28 g, 400 mmole), sodium iodide (24.72 g, 165 mmole), benzyl chloride (47.6 g, 376 mmole), acetone (412 ml) and methanol (412 ml) were taken into a reaction flask. The contents were heated to reflux and stirred for 5-6 hours. The solvents were removed completely under vacuum, followed by the addition of dichloromethane (300 ml) and washing of the organic layer with water. The solvent was distilled off completely under vacuum, n-hexane (220 ml) was added to the resulting oily mass, and then the mixture was stirred for 2-3 hours at 25-30° C. The material was filtered, washed with 50 ml of n-hexane, and then dried the solid to provide 124 g of methyl (±)-3-(2-benzyloxy-5-bromophenyl)-3-phenylpropionate (Yield: 88.5%; HPLC Purity: 99.53%).

### Step-3: Preparation of (±)-3-(2-Benzyloxy-5-bromophenyl)-3-phenylpropan-1-ol

Methyl (±)-3-(2-benzyloxy-5-bromophenyl)-3-phenylpropionate (100 g, 235 mmole), sodium borohydride (10.66 g, 282 mmole) and monoglyme (300 ml) were taken into a reaction flask. The reaction mixture was stirred for 10 minutes and cooled to 10° C. This was followed by the addition of aluminium chloride (15.633 g, 118 mmole) portion wise at below 10° C. over a period of 2 hours, and stirring for 1 hour at 10° C. Diluted hydrochloric acid was added drop wise to the reaction mass at below 5° C., followed by the addition of dichloromethane (900 ml), and stirring for 10 minutes. The layers were separated and the organic layer was washed with water, followed by distillation under vacuum to provide an oily mass. Petroleum ether (100 ml) was added to the oily mass and stirred for 1 hour at 25-30° C., then filtered and washed with 50 ml of petroleum ether. The material was dried at below 60° C. to give 88 g of (±)-3-(2-benzyloxy-5-bromophenyl)-3-phenylpropan-1-ol (Yield: 93.6%; HPLC Purity: 99.23%).

### Step-4: Preparation of (±)-Toluene-4-sulphonic acid 3-(2-benzyloxy-5-bromophenyl)-3-phenylpropyl ester

(±)-3-(2-Benzyloxy-5-bromophenyl)-3-phenylpropan-1-ol (100 g, 252 mmole), dichloromethane (500 ml), triethylamine (53 ml, 378 mmole) and p-toluenesulphonyl chloride (50.4 g, 265 mmole) were taken into a round bottom flask at 25-30° C. and stirred for 12 hours at 25-30° C. Water (250 ml) was added to the reaction mass and the pH of aqueous layer was adjusted to 3-4 with hydrochloric acid. The layers were separated and the organic layer was dried with sodium sulphate. The dichloromethane layer was concentrated under vacuum to provide 138 g of (±)-toluene-4-sulphonic acid 3-(2-benzyloxy-5-bromophenyl)-3-phenylpropyl ester as an oily residue (HPLC Purity: 95%).

### Step-5: Preparation of (±)—N,N-Diisopropyl-3-(2-benzyloxy-5-bromophenyl)-3-phenylpropylamine

### Method-A:

(±)-Toluene-4-sulphonic acid 3-(2-benzyloxy-5-bromophenyl)-3-phenylpropyl ester (100 g, 181.4 mmole) was dissolved in a mixture of acetonitrile (250 ml) and diisopropylamine (180 g, 1777 mmole), and the mixture was heated at 95-100° C. under closed conditions in an autoclave for 30 hours. The reaction mixture was cooled to 0° C. and stirred for 1 hour, followed by filtration and washing with acetonitrile (100 ml). Acetonitrile was distilled off completely under vacuum to provide an oily mass. Water (300 ml) was added to the oily mass and the pH was adjusted with hydrochloric acid to 1-2 to provide two layers. The oily layer was separated and dissolved into water (300 ml), and then washed with ether (200 ml). The aqueous layer was separated, and the pH was adjusted with ammonia solution to 9-10, followed by extraction with dichloromethane (300 ml). The dichloromethane was distilled off completely under vacuum, followed by the addition of isopropyl alcohol (80 ml) to the residue. The reaction mass was stirred for 8 hour at 25-30° C., and the solid was filtered, washed with isopropyl alcohol (40 ml) and then dried at 60° C. to provide 65 g of (±)—N,N-diisopropyl-3-(2-benzyloxy-5-bromophenyl)-3-phenylpropylamine (Purity by HPLC: 93.8%, Melting point: 52-56° C.).

### Step-6: Resolution of (±)—N,N-Diisopropyl-3-(2-benzyloxy-5-bromophenyl)-3-phenylpropyl amine

(±)—N,N-Diisopropyl-3-(2-benzyloxy-5-bromophenyl)-3-phenylpropylamine (100 g, 208 mmole) and isopropyl alcohol (1500 ml) were taken into a round bottom flask. Di-p-toluoyl-L-tartaric acid (80 g, 207 mmole) was added to the above mass and heated to reflux. The reaction mass was stirred for 1 hour at 86° C. and then slowly cooled to 25-30° C. After being stirred at 25-30° C. for 14 hours, the salts formed were filtered, washed with isopropyl alcohol and then dried to give 85 g of levorotatory salt [Melting point: about 120° C.; S.O.R: (−68°, C=1, Methanol)].

### Step-7: Preparation of (R)—N,N-Diisopropyl-3-(2-benzyloxy-5-bromophenyl)-3-phenylpropylamine

The levorotatory salt (85 g, obtained in step-6) was dissolved in water (425 ml) and basified with 2N sodium hydroxide solution (125 ml). The resulting solution was extracted with dichloromethane (425 ml), dried with sodium sulphate, and distilled under vacuum to give 40 g of (R)—N,N-diisopropyl-3-(2-benzyloxy-5-bromophenyl)-3-phenylpropylamine as a colourless oil. [Yield: 83%; [α]²⁴=−14° (C=5, ethanol, as on basis)].

### Step-8: (R)-4-Benzyloxy-3-(3-diisopropylamino-1-phenylpropyl)-benzoic acid

### Part-A: Preparation of (R)-4-Benzyloxy-3-(3-diisopropylamino-1-phenylpropyl)-benzoic acid hydrochloride

A mixture of magnesium (26 g), ethyl bromide (0.6 ml), iodine (2 crystals) and tetrahydrofuran (200 ml) was heated at 55-60° C. for initiation of reaction. To the resulting mass was added by drop wise addition a solution of (R)—N,N-diisopropyl-3-(2-benzyloxy-5-bromophenyl)-3-phenylpropylamine (100 g, 208.3 mmole) and ethyl bromide (54 ml) in tetrahydrofuran (500 ml). The reaction mixture was refluxed for 1 hour and cooled to −65° C. This was followed by the addition of powdered dry ice (100 g) at below −60° C. and stirring for 1 hour. Ammonium chloride solution (20%, 700 ml) was added to the reaction mixture at below 0° C. and stirred for 30 minutes. The layers were separated and the aqueous layer was washed with 100 ml of ether. The pH of aqueous layer was adjusted with diluted hydrochloric acid to 1-2 and the product was extracted with dichloromethane (300 ml). Dichloromethane was distilled off completely under vacuum to give a yellowish viscous oil. Acetone (85 ml) was added to the above oil and stirred for 14 hours at 25-30° C. to give (R)-4-Benzyloxy-3-(3-diisopropylamino-1-phenylpropyl)-benzoic acid hydrochloride as solid (Purity by HPLC: 99.68%).

### Part-B: Preparation of Amorphous Form of (R)-4-Benzyloxy-3-(3-diisopropylamino-1-phenylpropyl)-benzoic acid

A mixture of magnesium (26 g), ethyl bromide (0.6 ml), iodine (2 crystals) and tetrahydrofuran (200 ml) was heated at 55-60° C. for initiation of reaction. The resulting mass was followed by drop wise addition of a solution of (R)—N,N-diisopropyl-3-(2-benzyloxy-5-bromophenyl)-3-phenylpropylamine (100 g, 208.3 mmole) and ethyl bromide (54 ml) in tetrahydrofuran (500 ml). The reaction mixture was refluxed for 1 hour and cooled to −65° C. This was followed by the addition of powdered dry ice (100 g) at below −60° C. and stirred for 1 hour. Ammonium chloride solution (20%, 700 ml) was added to the reaction mixture at below 0° C. and stirred for 30 minutes. The layers were separated and the aqueous layer was washed with 100 ml of ether. The pH of aqueous layer was adjusted with diluted hydrochloric acid to 1-2 and the product was extracted with dichloromethane (300 ml). The dichloromethane layer was washed with 10% sodium bicarbonate solution and further washed with water (300 ml). Dichloromethane was distilled off completely under vacuum at 30-35° C. to give 100 g of amorphous (R)-4-benzyloxy-3-(3-diisopropylamino-1-phenylpropyl)-benzoic acid (Purity by HPLC: 96%).

### Part-C: Preparation of Crystalline Form of (R)-4-Benzyloxy-3-(3-diisopropylamino-1-phenylpropyl)-benzoic acid

Amorphous form of (R)-4-benzyloxy-3-(3-diisopropylamino-1-phenylpropyl)-benzoic acid (5 g, obtained in Part-B) was added to isopropyl alcohol (10 ml) and the mixture was heated at 70-75° C. to form a clear solution. The solution was cooled to 25-30° C. and stirred for 14 hours to produce 2.5 g of (R)-4-benzyloxy-3-(3-diisopropylamino-1-phenylpropyl)-benzoic acid as a crystalline form (Purity by HPLC: 99.5%).

### Step-9: Preparation of methyl (R)-4-benzyloxy-3-(3-diisopropylamino-1-phenylpropyl)-benzoate

Methanol (1000 ml) and (R)-4-benzyloxy-3-(3-diisopropylamino-1-phenylpropyl)-benzoic acid hydrochloride (100 g, 225 mmole) were taken into a round bottom flask. The contents were cooled to 10° C. followed by drop wise addition of thionyl chloride (48 ml). The reaction mixture was slowly heated and refluxed for 2-3 hours. Methanol was distilled off completely under vacuum to provide an oily mass, followed by the addition of 300 ml of dichloromethane. The dichloromethane layer was washed with saturated sodium bicarbonate solution (2×50 ml) followed by water washings. The dichloromethane solvent was distilled off completely under vacuum to give methyl (R)-4-benzyloxy-3-(3-diisopropylamino-1-phenylpropyl)-benzoate as an oily mass (Oil weight: 94 g). A mixture of isopropyl alcohol (250 ml) and water (250 ml) was added to the resulting oily mass and then stirred for 1 hour at 55-60° C. The resulting mass was cooled to 25-30° C. and stirred at 25-30° C. The separated solid was filtered, washed with a mixture of isopropyl alcohol (50 ml) and water (50 ml), and then the material was dried at 60° C. to give 75 g of methyl (R)-4-benzyloxy-3-(3-diisopropylamino-1-phenylpropyl)-benzoate (HPLC Purity: 99.52%).

### Step-10: Preparation of crystalline form of (R)-[4-benzyloxy-3-(3-diisopropylamino-1-phenylpropyl)-phenyl]-methanol

Methyl (R)-4-benzyloxy-3-(3-diisopropylamino-1-phenylpropyl)-benzoate (100 g, 218 mmole) in tetrahydrofuran (500 ml), and lithium aluminum hydride (8.7 g) were taken into a reaction flask and stirred for 14 hours at 25-30° C. The excess hydride was decomposed by the addition of ethyl acetate (200 ml) followed by the addition of water (100 ml). The resulting mass was filtered through a hyflo bed, and the bed was washed with tetrahydrofuran (50 ml), followed by complete distillation of tetrahydrofuran and ethyl acetate under vacuum at 50° C. to produce an oily mass. Petroleum ether (200 ml) was added to the oily mass followed by stirring for 18 hours at 25-30° C. The separated solid was filtered to give 20 g of (R)-[4-benzyloxy-3-(3-diisopropylamino-1-phenylpropyl)-phenyl]-methanol as a crystalline form (Purity by HPLC: 99.44%).

### Step-11: Preparation of (R)-2-(3-diisopropylamino-1-phenylpropyl)-4-hydroxy methylphenol

The crystalline form of (R)-[4-Benzyloxy-3-(3-diisopropylamino-1-phenylpropyl)-phenyl]-methanol (100 g, 232 mmole, obtained in step-10) and methanol (1000 ml) were taken into a Parhydrogenator. Palladium carbon (5%, 20 g) was added and the mixture was hydrogenated with 2-3 kg pressure at 50-55° C. until the completion of the reaction. The mixture was then filtered and the solvent was removed by vacuum at below 50° C. The resulting oil was dissolved in dichloromethane (100 ml) and the dichloromethane solution was washed with water, dried over sodium sulfate, and the solvent evaporated to give 78 g of a colorless oil. Ethyl acetate (70 ml) was added to the oil and the mixture was heated to 50-55° C. and stirred for 15 minutes. The resulting mass was cooled to 25-30° C., followed by drop wise addition of n-hexane (350 ml) at 25-30° C. The mixture was stirred for 3 hours at 25-30° C. The separated solid was filtered and washed with a mixture of ethyl acetate and n-hexane (25 ml:100 ml) and then dried the material at 45-50° C. to give 60 g of (R)-2-(3-diisopropylamino-1-phenylpropyl)-4-hydroxymethylphenol as a solid (Purity by HPLC: 99.5%).

### Step-12: Preparation of 2-[(1R)-3-[Bis(1-methylethyl)amino]-1-phenylpropyl]-4-hydroxymethylphenyl isobutyrate (Fesoterodine)

(R)-2-(3-Diisopropylamino-1-phenylpropyl)-4-hydroxymethylphenol (100 g, 292 mmole) was added to dichloromethane (2000 ml) and cooled to 0° C. This step was followed by the addition of a solution of isobutyryl chloride (31.1 g, 292 mmole) in dichloromethane (100 ml) at 0-5° C. over a period of 1 hour. The contents were stirred for 30 minutes followed by drop wise addition of a solution of triethyl amine (34.5 g, 297 mmole) in 50 ml of dichloromethane at 0-5° C. for 30 minutes. The resulting mass was stirred for 30 minutes, water (100 ml) was added to the mass, and then the layers were separated and the dichloromethane layer was washed with 5% sodium bicarbonate solution (100 ml). The dichloromethane layer was dried with sodium sulfate and then the dichloromethane was distilled off under vacuum to give 115 g of fesoterodine as oily mass (Yield: 95%).

### Step-13: Preparation of Fesoterodine Fumarate

A solution of fesoterodine (42 g) in methyl ethyl ketone (90 ml) was stirred with fumaric acid (12 g) at 80° C. for 1 hour. This was followed by the slow addition of cyclohexane (30 ml) under stirring and further stirred for 1 hour at 80° C. The solution was cooled slowly to 25-30° C. and stirred for 12 hours at the same temperature. The solution was further cooled at 0-5° C. and stirred for 12 hours. The separated solid was filtered and washed with a mixture of cyclohexane and methyl ethyl ketone to give fesoterodine fumarate (HPLC Purity: 99.88%).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. Solid state form of a fesoterodine intermediate selected from (R)-4-benzyloxy-3-(3-diisopropylamino-1-phenylpropyl)-benzoic acid and (R)-[4-benzyloxy-3-(3-diisopropylamino-1-phenylpropyl)-phenyl]-methanol, wherein:
a) the solid state form of (R)-4-benzyloxy-3-(3-diisopropylamino-1-phenylpropyl)-benzoic acid is an amorphous form characterized by the following properties:
i) a powder X-ray diffraction pattern showing no peaks substantially in accordance with FIG. 1;
ii) an IR spectrum substantially in accordance with FIG. 2;
iii) an IR spectrum having absorption bands at 3407, 2984, 2509, 1694, 1602, 1550, 1494, 1453, 1379, 1245, 1133, 1017, 785, 739, 699 and 651±1 cm⁻¹; and
iv) a differential scanning calorimetric (DSC) thermogram substantially in accordance with FIG. 3;
b) the solid state form of (R)-4-benzyloxy-3-(3-diisopropylamino-1-phenylpropyl)-benzoic acid is a crystalline form characterized by the following properties:
i) a powder X-ray diffraction pattern substantially in accordance with FIG. 4;
ii) a powder X-ray diffraction pattern having peaks at about 6.82, 9.27, 12.06, 13.44, 13.73, 14.88, 15.25, 15.62, 17.14, 17.45, 17.68, 19.92, 20.59, 21.83, 23.89, 24.30, 26.49, 26.72 and 27.06±0.2 degrees 2-theta;
iii) an IR spectrum substantially in accordance with FIG. 5; and
iv) an IR spectrum having absorption bands at about 3413, 3055, 3026, 2979, 1598, 1559, 1493, 1452, 1363, 1263, 1246, 1227, 1132, 1019, 931, 783, 751, 702 and 624±1 cm⁻¹; and
v) a differential scanning calorimetric (DSC) thermogram substantially in accordance with FIG. 6; and
c) the solid state form of (R)-[4-benzyloxy-3-(3-diisopropylamino-1-phenylpropyl)-phenyl]-methanol is a crystalline form characterized by the following properties:
i) a powder X-ray diffraction pattern substantially in accordance with FIG. 7;
ii) a powder X-ray diffraction pattern having peaks at about 7.07, 9.09, 9.76, 12.17, 12.43, 15.41, 15.71, 18.73, 19.66, 22.05, 22.32, 24.0, 24.57 and 24.84±0.2 degrees 2-theta;
iii) an IR spectrum substantially in accordance with FIG. 8;
iv) an IR spectrum having absorption bands at 3178, 3025, 2967, 2856, 2815, 1605, 1502, 1479, 1451, 1384, 1271, 1253, 1243, 1159, 1116, 1063, 1040, 1024, 803, 731, 708 and 691±1 cm⁻¹; and
v) a differential scanning calorimetric (DSC) thermogram substantially in accordance with FIG. 9.

2. A process for the preparation of the amorphous form of (R)-4-benzyloxy-3-(3-diisopropylamino-1-phenylpropyl)-benzoic acid as claimed in claim 1, comprising:
a) providing a solution of (R)-4-benzyloxy-3-(3-diisopropylamino-1-phenylpropyl)-benzoic acid in a chlorinated hydrocarbon solvent;
b) optionally, filtering the solution to remove extraneous matter; and
c) substantially removing the solvent from the solution to provide amorphous form of (R)-4-benzyloxy-3-(3-diisopropylamino-1-phenylpropyl)-benzoic acid.

3. The process of claim 2, wherein the chlorinated hydrocarbon solvent used in step-(a) is selected from the group consisting of methylene chloride, ethylene dichloride, chloroform, carbon tetrachloride, and mixtures thereof; wherein the solution in step-(a) is prepared by dissolving or extracting the (R)-4-benzyloxy-3-(3-diisopropylamino-1-phenylpropyl)-benzoic acid in the chlorinated hydrocarbon solvent at a temperature of 0° C. to the boiling temperature of the solvent used; wherein the solution obtained in step-(a) is optionally subjected to carbon treatment or silica gel treatment; and wherein the removal of solvent in step-(c) is accomplished by substantially complete evaporation of the solvent, concentrating the solution or distillation of solvent under inert atmosphere, spray drying, vacuum drying, agitated thin-film (ATFD) drying, or a combination thereof.

4. The process of claim 3, wherein the chlorinated hydrocarbon solvent is methylene chloride; and wherein the (R)-4-benzyloxy-3-(3-diisopropylamino-1-phenylpropyl)-benzoic acid in step-(a) is dissolved or extracted in the chlorinated hydrocarbon solvent at a temperature of about 25° C. to about 80° C.

5. A process for the preparation of the crystalline form of (R)-4-benzyloxy-3-(3-diisopropylamino-1-phenylpropyl)-benzoic acid as claimed in claim 1, comprising:
a) providing a solution of (R)-4-benzyloxy-3-(3-diisopropylamino-1-phenylpropyl)-benzoic acid in an alcohol solvent;
b) optionally, filtering the solution to remove extraneous matter; and
c) isolating and/or recovering crystalline form of (R)-4-benzyloxy-3-(3-diisopropylamino-1-phenylpropyl)-benzoic acid from the solution.

6. The process of claim 5, wherein the alcohol solvent used in step-(a) is selected from the group consisting of methanol, ethanol, n-propanol, isopropyl alcohol, isobutanol, n-butanol, tert-butanol, amyl alcohol, isoamyl alcohol, and mixtures thereof; wherein the solution in step-(a) is prepared by dissolving or extracting the (R)-4-benzyloxy-3-(3-diisopropylamino-1-phenylpropyl)-benzoic acid in the alcohol solvent at a temperature of 0° C. to the boiling temperature of the solvent used; wherein the solution obtained in step-(a) is optionally subjected to carbon treatment or silica gel treatment; and wherein the isolation of pure crystalline form of (R)-4-benzyloxy-3-(3-diisopropylamino-1-phenylpropyl)-benzoic acid in step-(c) is carried out by forcible crystallization or spontaneous crystallization.

7. The process of claim 6, wherein the alcohol solvent is isopropyl alcohol; wherein the (R)-4-benzyloxy-3-(3-diisopropylamino-1-phenylpropyl)-benzoic acid is dissolved or extracted in the alcohol solvent at about 50° C. to about 80° C.; wherein the crystallization in step-(c) is initiated by cooling, seeding, partial removal of the solvent from the solution, by adding an anti-solvent to the solution, or a combination thereof; and wherein the recovering in step-(c) is carried out by filtration, filtration under vacuum, decantation, centrifugation, filtration employing a filtration media of a silica gel or celite, or a combination thereof.

8. The process of claim 7, wherein the crystallization is carried out by cooling the solution while stirring at a temperature of about 0° C. to about 30° C. for about 30 minutes to about 20 hours.

9. A process for the preparation of the crystalline form of (R)-[4-benzyloxy-3-(3-diisopropylamino-1-phenylpropyl)-phenyl]-methanol as claimed in claim 1, comprising:
a) reacting methyl (R)-4-benzyloxy-3-(3-diisopropylamino-1-phenylpropyl)-benzoate with a reducing agent, optionally in the presence of a Lewis acid, in a first solvent to produce a first reaction mass containing (R)-[4-benzyloxy-3-(3-diisopropylamino-1-phenylpropyl)-phenyl]-methanol;
b) optionally, subjecting the first reaction mass to work up methods selected from the group consisting of a filtration, a washing, an extraction, an acid/base treatment, an evaporation, or a combination thereof;
c) substantially removing the first solvent from the first reaction mass to provide an oily mass containing (R)-[4-benzyloxy-3-(3-diisopropylamino-1-phenylpropyl)-phenyl]-methanol;
d) combining the oily mass with a second solvent to produce a second reaction mass; and
e) recovering crystalline form of (R)-[4-benzyloxy-3-(3-diisopropylamino-1-phenylpropyl)-phenyl]-methanol from the second reaction mass.

10. The process of claim 9, wherein the reducing agent used in step-(a) is selected from the group consisting of lithium aluminium hydride, sodium borohydride, sodium cyanoborohydride and sodium triacetoxyborohydride; wherein the Lewis acid used in step-(a) is selected from the group consisting of aluminium chloride, calcium chloride, boron triflouride and zinc chloride; wherein the first solvent used in step-(a) is selected from the group consisting of monoglyme, diglyme, tetrahydrofuran, ethers, and mixtures thereof; and wherein the second solvent used in step-(d) is selected from the group consisting of n-pentane, n-hexane, n-heptane, n-octane, petroleum ether, cyclohexane, and mixtures thereof.

11. The process of claim 10, wherein the removal of solvent in step-(c) is accomplished by substantially complete evaporation of the solvent, concentrating the solution or distillation of solvent under inert atmosphere, or a combination thereof; wherein the combining of the oily mass with the second solvent in step-(d) is accomplished either by adding the oily mass to the second solvent or by adding the second solvent to the oily mass; wherein the reaction mass obtained after completion of the addition process in step-(d) is stirred at a temperature of about 0° C. to about 50° C. for at least 30 minutes; and wherein the recovering in step-(e) is carried out by filtration, filtration under vacuum, decantation, centrifugation, filtration employing a filtration media of a silica gel or celite, or a combination thereof.

12. The process of claim 11, wherein the addition in step-(d) is carried out at a temperature of about 20° C. to about 80° C.; and wherein the reaction mass obtained after completion of the addition process in step-(d) is stirred at about 10° C. to about 45° C. for about 1 hour to about 25 hours.

13. A process for the preparation of highly pure fesoterodine or a fumarate salt thereof using the crystalline form of (R)-[4-benzyloxy-3-(3-diisopropylamino-1-phenylpropyl)-phenyl]-methanol as claimed in claim 1, comprising:
a) hydrogenation the crystalline form of (R)-[4-benzyloxy-3-(3-diisopropylamino-1-phenylpropyl)-phenyl]-methanol using palladium carbon catalyst in the presence of hydrogen gas in methanol to produce (R)-2-(3-diisopropylamino-1-phenylpropyl)-4-hydroxymethylphenol;
b) condensing the (R)-2-(3-diisopropylamino-1-phenylpropyl)-4-hydroxymethylphenol obtained in step-(a) with isobutyryl chloride in dichloromethane to produce fesoterodine; and
c) treating the fesoterodine obtained in step-(b) with fumaric acid to produce highly pure fesoterodine fumarate salt.
